SEQUENCE LISTING (19 sequences)

SEQ ID NO: 1 - protein, 60 aa, synthetic construct
MPKKSKVTITDIARRVNMTTITVSRALTKPELVKKETLDRILEVARELNYHPNANARALA

SEQ ID NO: 2 - protein, 57 aa, synthetic construct
MKPVTLYDVAEYAGVSYQTVSRVVNQASHVSAKTREKVEAAMAELNYIPNRVAQQLA

SEQ ID NO: 3 - protein, 59 aa, synthetic construct
MNKPQITIKDIARALNVSPSTVSRALKDNPDISKETRDLVHAYAREHNYIPNRVAQQLA

SEQ ID NO: 4 - protein, 59 aa, synthetic construct
QQTTETVGLVVGDVSDPFFGAMVKAVEQVAYHTGNFLLIGNGYHNEQKERQAIEQLIRH

SEQ ID NO: 5 - protein, 60 aa, synthetic construct
GKQSLLIGVATSSLALHAPSQIVAAIKSRADQLGASVVVSMVERSGVEACKAAVHNLLAQ

SEQ ID NO: 6 - protein, 60 aa, synthetic construct; identical to SEQ ID NO: 5

SEQ ID NO: 7 - protein, 58 aa, synthetic construct
RCAALVVHAKMIPDADLASLMKQMPGMVLINRILPGFENRCIALDDRYGAWLATRHLI

SEQ ID NO: 8 - protein, 58 aa, synthetic construct
RVSGLIINYPLDDQDAIAVEAACTNVPALFLDVSDQTPINSIIFSHEDGTRLGVEHLV

SEQ ID NO: 9 - protein, 58 aa, synthetic construct; identical to SEQ ID NO: 8

SEQ ID NO: 10 - protein, 60 aa, synthetic construct
QQGHTRIGYLCSNHSISDAEDRLQGYYDALAESGIAANDRLVTFGEPDESGGEQAMTELL

SEQ ID NO: 11 - protein, 58 aa, synthetic construct
ALGHQQIALLAGPLSSVSARLRLAGWHKYLTRNQIQPIAEREGDWSAMSGFQQTMQML

SEQ ID NO: 12 - protein, 58 aa, synthetic construct; identical to SEQ ID NO: 11

SEQ ID NO: 13 - protein, 60 aa, synthetic construct
GRGRNFTAVACYNDSMAAGAMGVLNDNGIDVPGEISLIGFDDVLVSRYVRPRLTTVRYPI

SEQ ID NO: 14 - protein, 60 aa, synthetic construct
NEGIVPTAMLVANDQMALGAMRAITESGLRVGADISVVGYDDTEDSSCYIPPLTTIKQDF

SEQ ID NO: 15 - protein, 60 aa, synthetic construct; identical to SEQ ID NO: 14

SEQ ID NO: 16 - protein, 51 aa, synthetic construct
VTMATQAAELALALADNRPLPEITNVFSPTLVRRHSVSTPSLEASHHATSD

SEQ ID NO: 17 - protein, 59 aa, synthetic construct
RLLGQTSVDRLLQLSQGQAVKGNQLLPVSLVKRKTTLAPNTQTASPRALADSLMQLARQ

SEQ ID NO: 18 - protein, 42 aa, synthetic construct
RLLGQTSVDRLLQLSQGQAVKGNQLLPVSLVKRKTTLAPNTQ

SEQ ID NO: 19 - protein, 8 aa, synthetic construct
VSRLESGQ

(19) United States
(12) Patent Application Publication
(10) Pub. No.: US 2023/0064064 A1
(43) Pub. Date: Mar. 2, 2023

(54) METHODS FOR ROBUST DESIGN OF MODULAR REPRESSORS

(71) Applicants: UNIVERSITY OF NORTH TEXAS, Dallas, TX (US); The University of Texas System, Austin, TX (US)

(72) Inventors: Clement T. Y. CHAN, DALLAS, TX (US); Faruck MORCOS, Austin, TX (US); Xian-Li JIANG, Austin, TX (US)

(21) Appl. No.: 17/823,921

(22) Filed: Aug. 31, 2022

Related U.S. Application Data
(60) Provisional application No. 63/239,381, filed on Aug. 31, 2021.

(51) Int. Cl. C12N 15/63; G16B 20/30

(52) U.S. Cl. CPC C12N 15/635; G16B 20/30

(57) ABSTRACT

A method can include receiving a protein sequence (S) of a hybrid repressor, determining an original compatibility score C(S), where the compatibility score C is a function of the protein sequence (S) and predicting, based on the compatibility score C, a performance of the hybrid repressor. The hybrid protein sequence includes a plurality of DNA-binding modules (DBMs) and a plurality of ligand-binding modules (LBMs).

### CROSS-REFERENCE TO RELATED APPLICATIONS

This application claims priority to U.S. Provisional Application Ser. No. 63/239,381, entitled, “Coevolutionary Methods enable Robust Design of Modular Repressors by Reestablishing Intra-protein Interactions,” filed on Aug. 31, 2021, by Chan, et al., which is incorporated herein by reference in its entirety.

### STATEMENT REGARDING FEDERALLY SPONSORED RESEARCH OR DEVELOPMENT

This invention was made with government support under contracts R35GM133631 and 1R15GM135813-01, both awarded by The National Institutes of Health—NIH. The government has certain rights in the invention.

### INCORPORATION BY REFERENCE OF MATERIAL IN AN XML FILE

This application incorporates by reference the Sequence Listing contained in the following XML, file being submitted concurrently herewith: File name: 4752-01501_ChanC1_Sequence listing.xml; created on Aug. 31, 3022; and having a files size of 18 KB. The information in the Sequence Listing is incorporated herein in its entirety for all purposes.

### TECHNICAL FIELD

The present disclosure relates generally to transcriptional repressors. More particularly, the present disclosure relates to methods and compositions for the generation of high performance hybrid transcriptional repressors.

### BACKGROUND

A synthetic genetic circuit is a set of genetic parts, including both coding and regulatory DNA, that are delivered into an organism and together carry out a desired function. Synthetic genetic circuit approaches represent programmable ways to create distinct signal response behavior in cells and organisms. However, the construction of genetic circuits is constrained by the lack of modular components; in natural biological systems, a biological sensor usually responds to a unique molecular signal to control gene expression driven by a specific genetic element, such as a promoter. This rigidity poses a challenge for scientists to implement diverse circuit designs in biological systems.

Genetic sensors with unique combinations of DNA recognition and allosteric response can be created by hybridizing DNA-binding modules (DBMs) and ligand-binding modules (LBMs) from distinct transcriptional repressors. This module swapping approach is limited by incompatibility between DBMs and LBMs from different proteins, due to the loss of critical module-module interactions after hybridization.

An ongoing need exists for novel modular components for applications directed to the elucidation and manipulation of signal response behavior in biological systems.

### BRIEF DESCRIPTION OF DRAWINGS

For a detailed description of the aspects of the disclosed processes and systems, reference will now be made to the accompanying drawings in which:

FIG. 1a depicts a set of DNA-binding modules (DBMs) and ligand-binding modules (LBMs) interactions based on coevolutionary cues among LacI family members.

FIG. 1b depicts a heatmap showing the ΔC(S) scores of all LacI-RbsR candidates with a single mutation at its LBM relative to the score for original repressor.

FIG. 1c illustrates the C(S) scores of double mutants as function of amino acid residue for LacI-RbsR candidates.

FIG. 1d is a graph of the C(S) scores triple mutants and plotted the ΔC(S) scores relative to the best single mutant and best double mutant, respectively for LacI-RbsR candidates.

FIGS. 2a-2h are plots of the compatibility scores of the mutated hybrid repressor candidates relative to the compatibility score of the original hybrid repressor and heatmap.

FIGS. 3a-3d depict fold change in induction for the mutated hybrid repressor candidates compared to an original hybrid repressor for the following samples (a) LacI-RbsR, (b) PurR-GalR, (c) CelR-RbsR, and (d) RbsR-GalR.

FIG. 4 depicts flow cytometric data from a transcriptional reporter assay for the indicated samples.

FIG. 5 depicts a crystal structure of native LacI in complex with its DNA operator and indicates homologous positions on LacI for mutations that rescue hybrid repressors.

FIGS. 6a-6d depict the fold change in induction from experimentally tested mutant candidates of hybrid repressors, including (a) LacI-RbsR, (b) PurR-GalR, (c) CelR-RbsR, (d) RbsR-GalR, (e) RbsR-LacI, (f) XltR-GalR, (g) MalR-LacI, and (h) XltR-ScrR.

FIGS. 7a and 7b depict sequence alignments of the indicated molecules.

FIGS. 8a-8d present in tabular form the amino acid residues that are the most incompatible with the mutation for the indicated candidate.

FIGS. 9a and 9b are exemplary forward versus side scatter log area plots for samples subjected to flow cytometric analysis.

### DETAILED DESCRIPTION

Disclosed herein is a novel module swapping strategy, designated MSS, which may be used to produce modular biosensors that function as allosterically regulated transcription repressors. In an aspect, the allosterically regulated transcription repressor is a hybrid repressor comprising an N-terminal domain that interacts with promoters (DNA-binding) and a C-terminal domain that senses molecular signals (ligand-binding). The hybrid repressor may regulate transcription by binding to a promoter in response to ligands that are permeable to most types of cells. Without wishing to be limited by theory, hybrid repressors can facilitate flexible connections from chemical signals to promoters for controlling gene expression. For example, modular repressors (e.g., allosterically regulated transcription repressors) can implement circuit topologies that require multiple signaling molecules to activate the expression of an output gene, or to use one signal to induce gene expression driven by different promoters. In summary, an MSS may be a tool for generating molecules that can positively impact bioengineering in a broad range of directions.

One major challenge in creating hybrid repressors is that some DBMs and LBMs are incompatible. Herein the compatibility refers to the extent to which the hybrid repressor comprising a DBM and LBM is able to carry out its intended function (e.g., transcriptional repression) when compared to a reference repressor. Incompatibility between a DBM and LBM may lead to the poor performance of some hybrid repressors, thereby impeding the linkage between many inputs and outputs.

In one or more aspects, an MSS of the present disclosure is a robust strategy for modifying poorly functional hybrid repressors in order to rescue their activities. An MSS of the type disclosed herein may include rational protein engineering based on coevolutionary traits within a protein family, compatibility score assignment, identification of potential candidates, preparation of potential candidates, and comparison of the candidates function to a reference material.

In an aspect, the MSS is used to create modular biosensors which are allosterically regulated transcription repressors. Herein a transcriptional repressor refers to proteins that bind to specific sites on DNA and prevent transcription of nearby genes. At the cellular level, transcriptional repression is mediated by a diverse collection of molecular signaling pathways. A pervasive mechanism of signaling in these pathways is allosteric regulation, in which binding of a ligand induces a conformational change in some target molecule, triggering a signaling cascade.

Allosteric transcription factors can be induced or corepressed by binding to a ligand. An allosteric transcription factor can adopt multiple conformational states, each of which has its own affinity for the ligand and for its DNA target site. In an aspect, the MSS is utilized in the production of a hybrid repressor wherein at least a portion of the repressor has a sequence homologous to sequences common to the lac repressor family of proteins. Herein the lac repressor protein refers to a DNA-binding protein that inhibits the expression of genes coding for proteins involved in the metabolism of lactose in bacteria and termed LacI. Repressors in the LacI families are composed of two discrete, conserved and potentially interchangeable modules that are responsible for the detection of ligands and for interaction with DNA-based promoters.

Herein the homology of portions of the hybrid repressor to the original repressor (e.g., LacI) may range from about 30% to about 80%.

Without wishing to be limited by theory, the loss of critical module-module interactions in a hybrid repressor is a major cause of reduced protein activities. The methods of the present disclosure (i.e., MSS) overall approach is to predict mutations that may restore native-like interactions interrupted by the hybridization process between different repressors. The prediction may be carried out utilizing a computational model to study interactions between LBMs and DBMs. In an aspect, the model is based on the assumption that a network of evolutionary relevant DBM-LBM couplings are essential for allosteric protein function and thus, these interactions are highly coevolving during the history of the protein family. As a result, if an amino acid residue involved in such a network is changed during evolution, its interacting amino acid residue needs to coevolve to maintain the function. In one or more aspects, the hybrid repressor is a LacI homolog. In such aspects, computational analysis is carried out on equal to or greater than about 50,000 homologs, alternatively equal to or greater than about 75,000 homologs, alternatively equal to or greater than about 100,000 analogs or from about 50,000 homologs to about 100,000 homologs to determine the interaction between the DBM and LBM. In an aspect, the computational analysis is carried out on over 70,000 LacI homologs to identify inter-modular residue pairs that coevolve and then utilize these pairs to compute a compatibility score C(S) where S represents a given hybrid protein sequence that consists of different LBMs and DBMs, which successfully inferred the performance of hybrid repressors.

In an aspect, the MSS comprises a coevolutionary modeling approach to engineer mutations within LBMs that can restore desirable module-module interactions for rescuing hybrid repressor activities. Without wishing to be limited by theory, some residues involved in module-module interactions should not be altered because they also play key roles in interacting with other residues within the LBM to maintain structural integrity.

In an aspect, the MSS comprises a DBM-LBM compatibility model to predict mutations that are expected to improve functionality of the hybrid repressor. A compatibility score C(S) for a hybrid repressor may be computed using an inter-modular coevolutionary coupling strength parameters, eij (Ai, Aj), inferred from multiple sequence alignments. For example, the C(S) for a hybrid repressor may be computed using an inter-modular coevolutionary coupling strength parameters, eij (Ai, Aj), inferred from multiple sequence alignments of LacI homologs using global inference of the joint distribution of sequences in the family. For a given hybrid sequence, a mutation at residue i updates all parameters, eij (Ai, Aj), which describe interactions of the mutated residue, i, with all of its coevolving partners, j, resulting in a change in C(S) score. In an aspect, the C(S) score of a hybrid repressor produced using an MSS of the type disclosed herein ranges from about −90 to about −30.

In one or more aspects, an MSS of the present disclosure further comprises testing the repressor activity of a candidate hybrid repressor. Any suitable methodology may be used for testing the activity of the candidate hybrid repressor. For example, each candidate hybrid repressor may be characterized with an in vivo transcriptional assay using Escherichia coli cells. Specifically, the candidate hybrid repressor may be constitutively expressed in cells to repress the expression of green fluorescent protein, GFP. Activities of allosteric response and transcriptional regulation may be assessed by comparing GFP levels in cells that were exposed and unexposed to the corresponding inducer of the target repressor. In an aspect, a candidate hybrid repressor prepared using a MSS of the type disclosed herein has an activity that is increased by greater than about 5 fold, alternatively greater than about 10 fold, alternatively greater than about 100 fold, alternatively greater than about 250 fold or alternatively form about 5 fold to about 500 fold when compared with a nonhybrid reference.

Genetic sensors with unique combinations of DNA recognition and allosteric response can be created by hybridizing DBMs and LBMs from distinct transcriptional repressors. Disclosed herein is a design strategy for restoring key interactions between DBMs and LBMs by using a computational model informed by coevolutionary traits in the LacI family. This model predicts the influence of proposed mutations on protein structure and function, quantifying the feasibility of each mutation for rescuing hybrid repressors. An MSS of the type disclosed herein may accurately predict which hybrid repressors can be rescued by mutating residues to reinstall relevant module-module interactions. Utilization of the MSS enhances the molecular and mechanistic understanding of LacI family proteins, and advances the ability to design modular genetic parts.

### EXAMPLES

The presently disclosed subject matter having been generally described, the following examples are given as particular aspects of the subject matter and to demonstrate the practice and advantages thereof. It is understood that the examples are given by way of illustration and are not intended to limit the specification or the claims in any manner.

In this study, the presently disclosed DBM-LBM compatibility model (i.e., MSS) was harnessed to predict mutations that are expected to improve functionality of hybrid repressors. A compatibility score C(S) for a hybrid repressor was computed using the inter-modular coevolutionary coupling strength parameters, eij (Ai, Aj), inferred from multiple sequence alignment of LacI homologs using global inference of the joint distribution of sequences in the family.

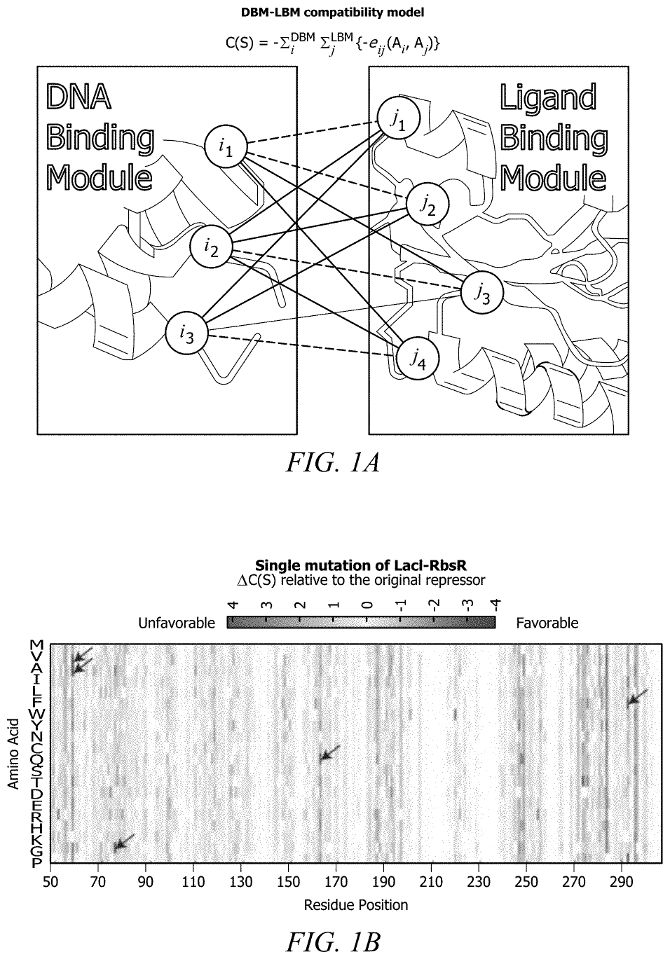

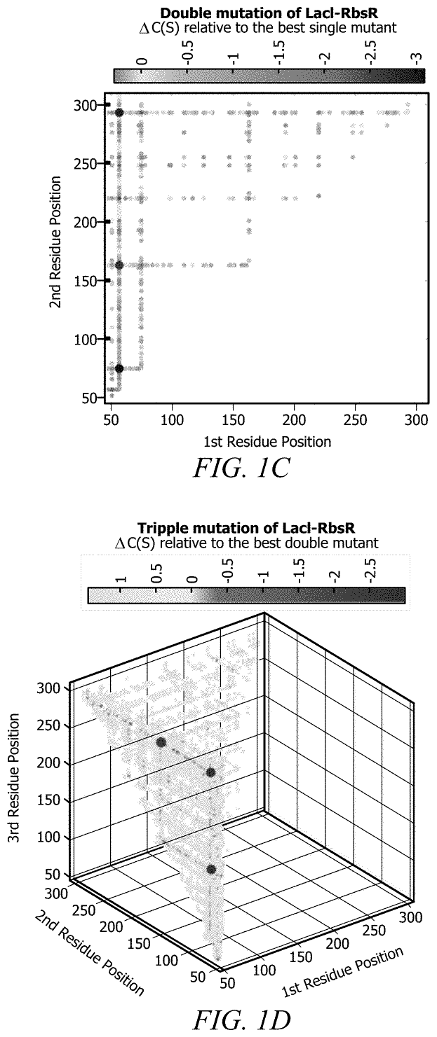

For a given hybrid sequence, a mutation at residue i updates all parameters, eij (Ai, Aj), which describe interactions of the mutated residue, i, with all of its coevolving partners, j, resulting in a change in C(S) score (FIG. 1a). Using this computational model, C(S) scores were systematically computed for mutations at LBMs. Mutations at DBMs were not considered because these modules are small (approximately 47 amino acid residues) and many residues are directly involved in DNA binding and recognition; mutating a DBM is likely to affect DNA binding properties of the protein. Mutants with the best C(S) scores were then selected, which represented candidates for improving repressor activities.

To test whether utilization of an MSS of the type described herein can rescue hybrid repressors, the strategy was applied to 8 hybrid repressors. A total of 35 hybrid repressors was generated; 18 of them were highly efficient and produce a dynamic range of induction over 10-fold; another 8 hybrids were poorly functional, which generated significantly reduced dynamic range of induction (3- to 10-fold); and the last 7 hybrids have no significant activities (induction is less than 3-fold). For the 8 cases with reduced activities, the fact that these hybrids still have some biological activity suggested that the LBM and the DBM can still interact to some extent; however, for those other 7 hybrids with no activities, all module-module interactions could be completely lost.

The 8 repressors with reduced activities were selected for modification using the MSS because these repressors may represent cases where a majority of essential LBM-DBM interactions are maintained and only a few interacting pairs are disrupted; therefore, only a few mutations may be necessary to fully restore repressor activities. The hybrid repressor, LacI-RbsR, which generates a 5-fold increase of induction in response to its inducer, ribose was analyzed first. This hybrid repressor contained a LacI DBM and a RbsR LBM (all hybrids in this study are named following this pattern).

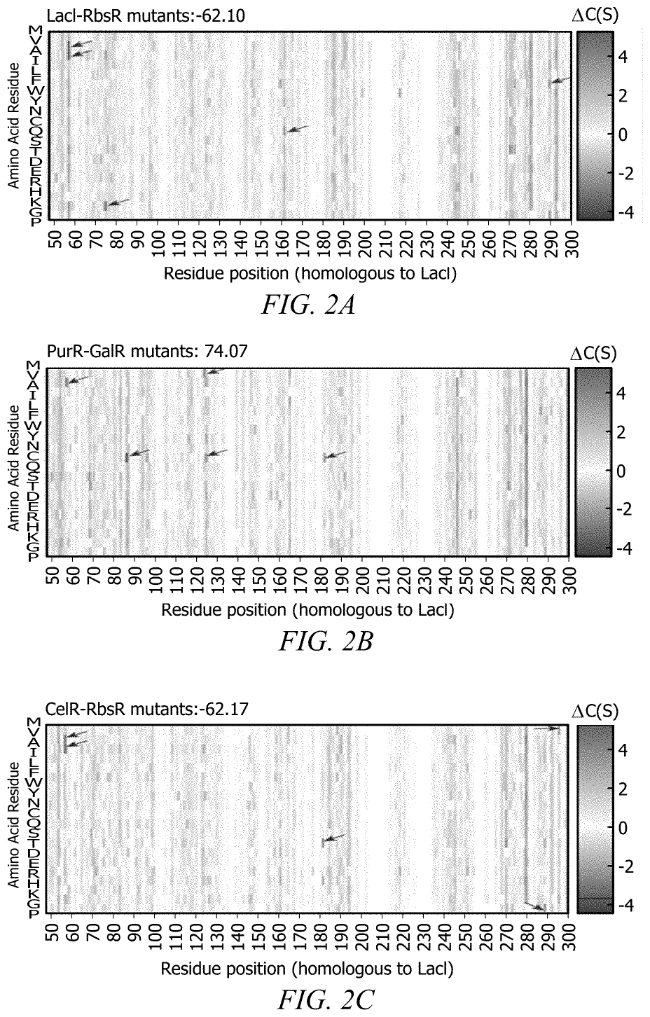

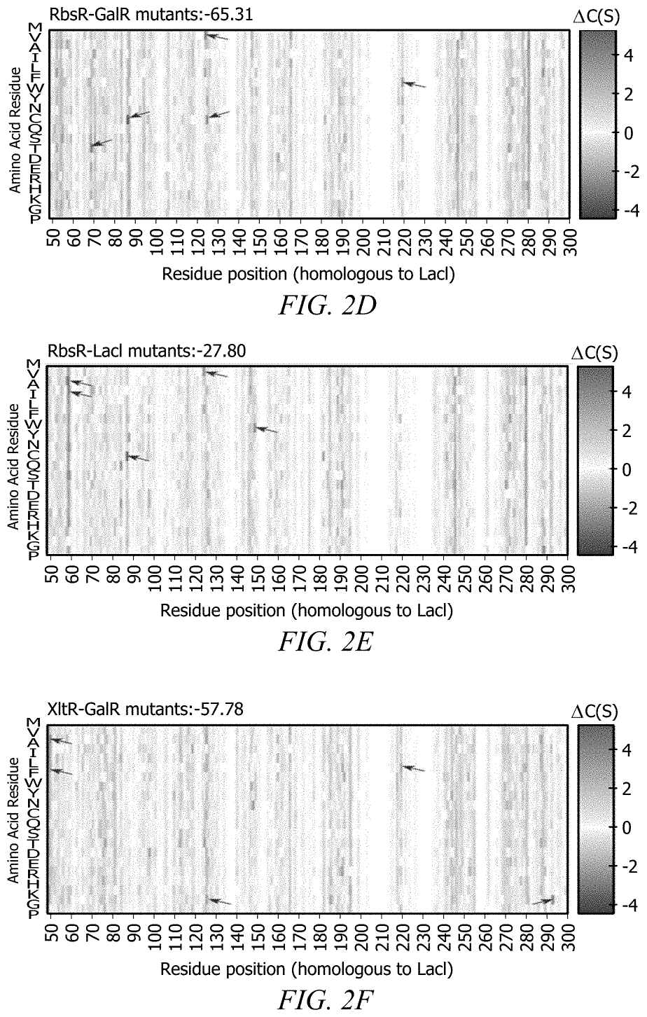

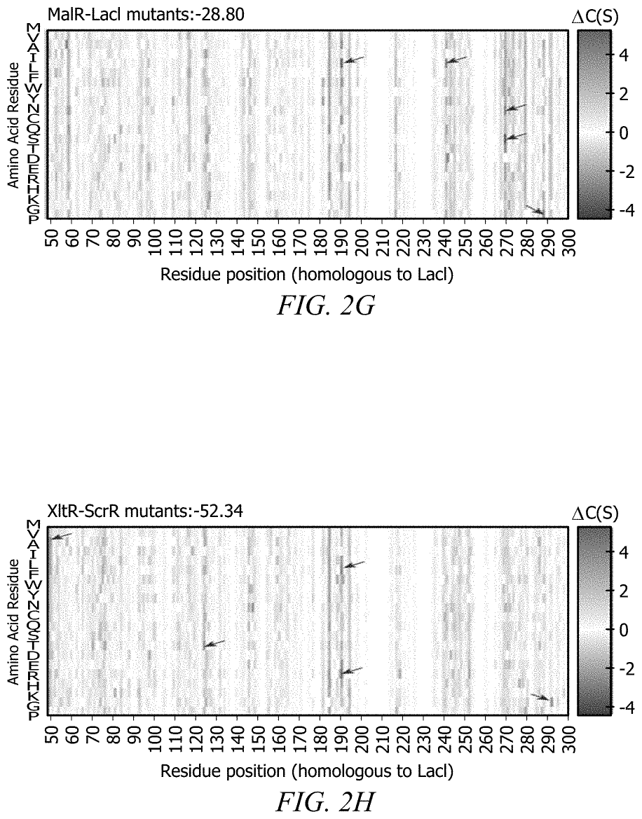

A heatmap is shown in FIG. 1b to illustrate the effect on the compatibility score of all possible single mutations for the LacI-RbsR hybrid repressor. Among the mutations that improve the compatibility score, the top 5 favorable mutations were K57V, K57A, F75G, N163Q, and K295F. Mutations at the LBM were the only one considered, therefore the mutational effects on compatibility scores are additive since the model only analyzes coevolutionary cues between DBMs and LBMs. Consequently, mutants with two and three of these favorable mutations lead to a further improved compatibility score. Therefore, the top double mutations involve the same residue positions (57, 75, 163, and 295) as found in the single mutation profile (FIG. 1b). In the triple mutation profile, residue positions 57, 75, 163, and 295 were also involved in the top 2 mutants (FIG. 1c). After analyzing LacI-RbsR, this approach was used to study other hybrid repressors with similar performance. A total of 8 hybrids, which originally only generated 3-10 folds induction in gene expression were investigated: LacI-RbsR, PurR-GalR, CelR-RbsR, RbsR-GalR, RbsR-LacI, CelR-RbsR, XltR-GalR, MalR-LacI, and XltR-ScrR (FIG. 2). Triple mutations for each hybrid repressor were predicted that possess the best C(S) score.

Hybrid repressors can be designed to improve allosteric regulation activities. The coevolutionary modeling approach (i.e., MSS) then investigated to determine if the methodology was sufficient to rationally improve the performance of hybrid repressors. For all eight hybrid repressors, two triple mutation candidates with top C(S) scores were selected for experimental characterization. In all eight cases, the two 3-mutation candidates have two shared mutations; within the three mutations of each candidate, all possible combinations with single, double and triple mutants were considered, which led to a set of 11 mutants for each hybrid repressor. The additive effect of multiple mutations on C(S) score ensured the single/double mutations were also among the top candidates in their respective cohort (FIGS. 1b to 1d).

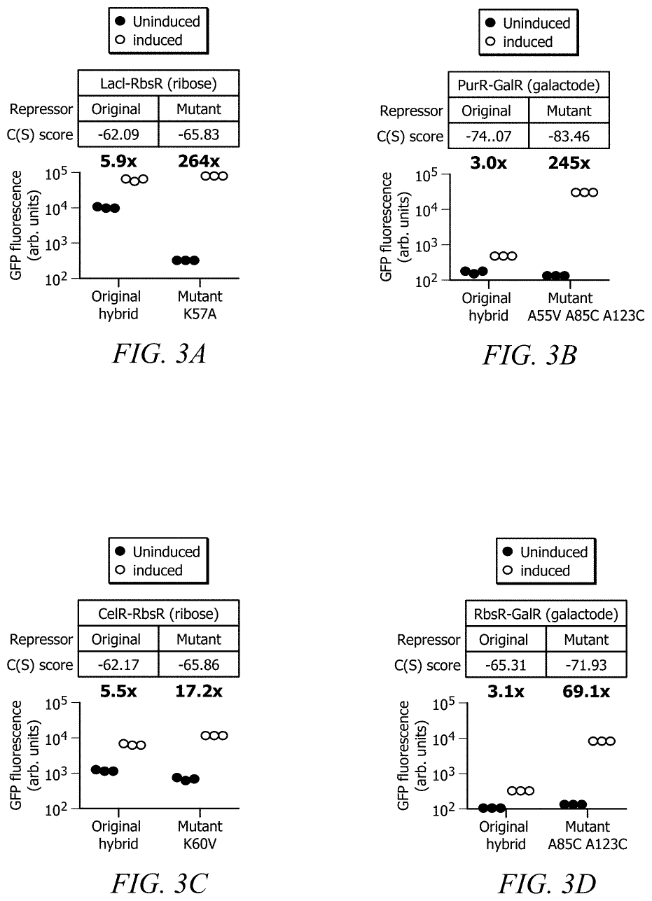

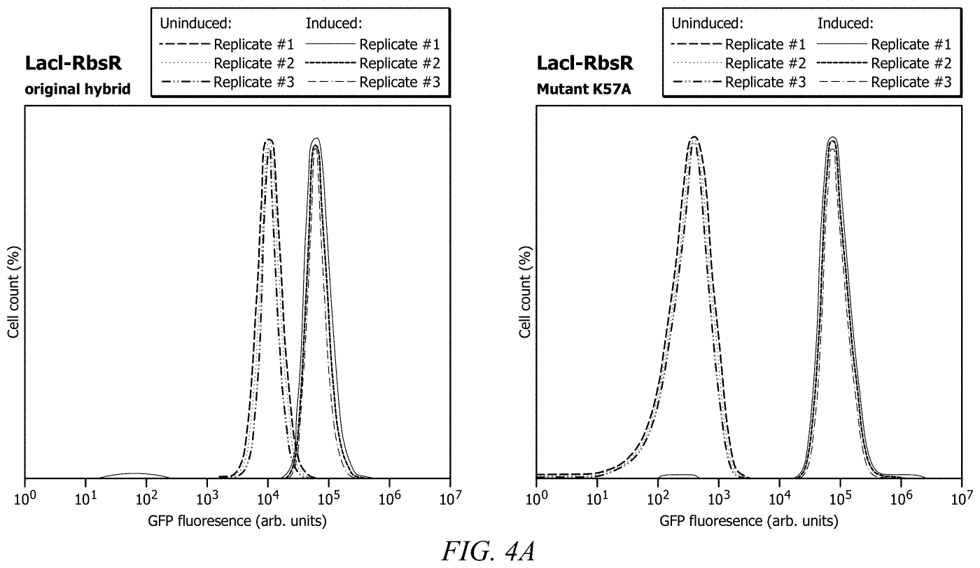

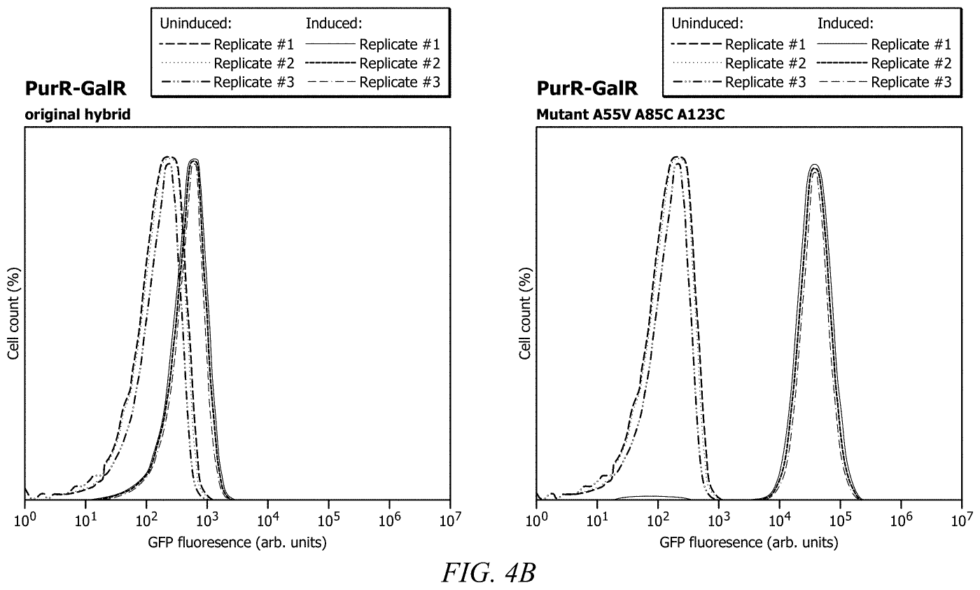

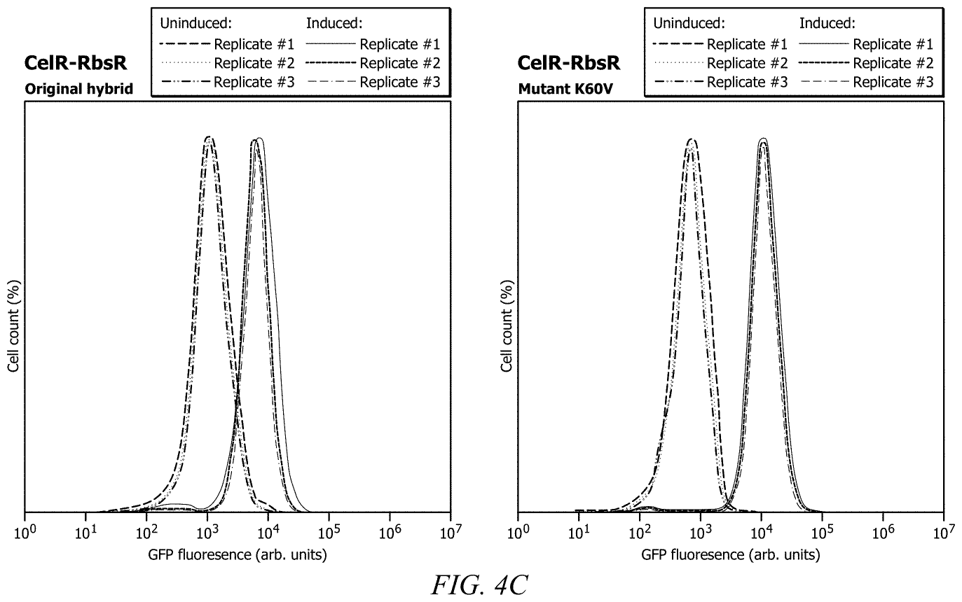

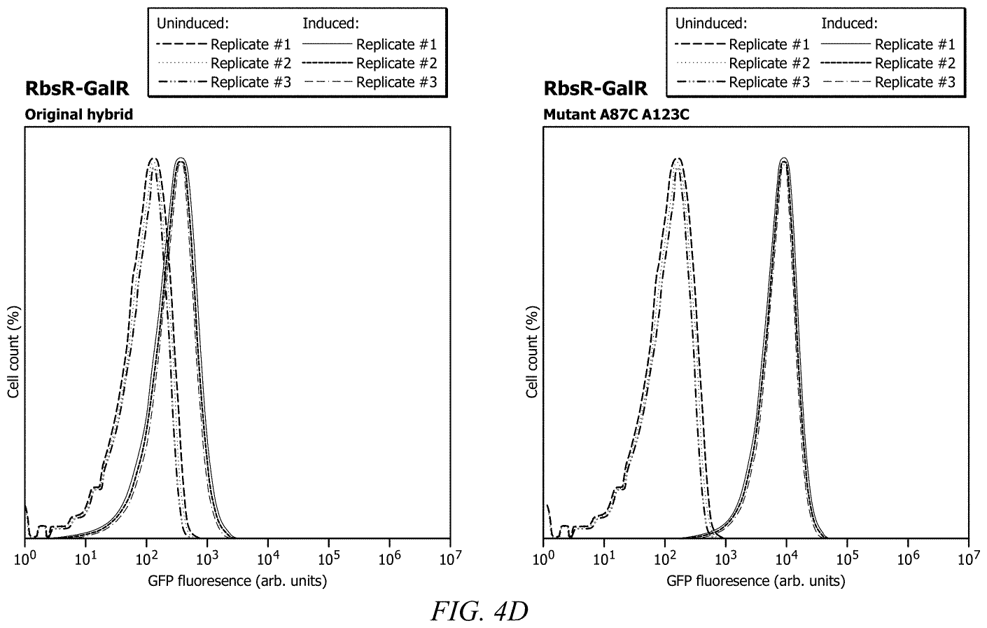

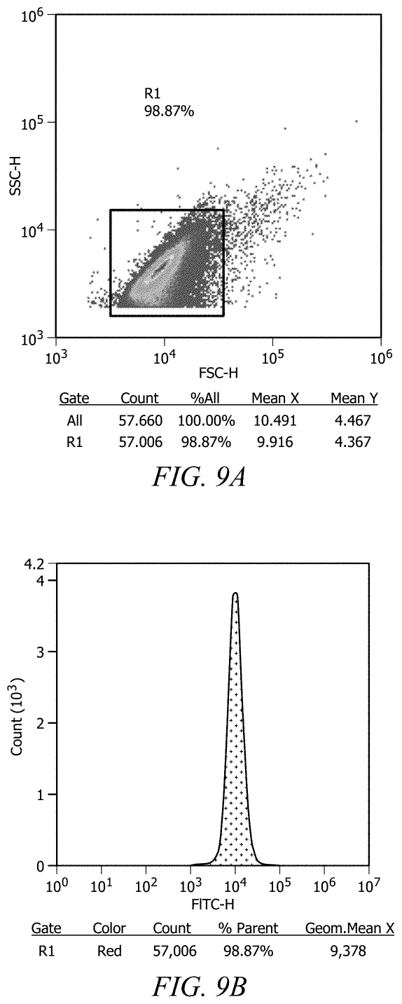

Each mutant was characterized with an in vivo transcriptional assay using Escherichia coli cells where the repressor was constitutively expressed in cells to repress the expression of GFP. The conditions for flow cytometric analysis were determined and a representative example of the gating strategy is presented in FIG. 9 using the data from FIG. 2. With reference to FIG. 9(a) Cells were gated using a forward versus side scatter log area plot, aiming to eliminate multi-cell aggregates. The percentage and total number of cells within the gate is shown. As shown in FIG. 9(b), the distribution of GFP fluorescence signal from these gated cells are shown in the histogram. Activities of allosteric response and transcriptional regulation were assessed by comparing GFP levels in cells that were exposed and unexposed to the corresponding inducer of the target repressor. Characterization data from all 88 mutants from the 8 hybrid repressors was collected. The MSS predicted mutations significantly improved activities of four hybrid repressors, such that they became capable to generate GFP signal inductions that were above 10 folds. These modified repressors include LacI-RbsR, PurR-GalR, CelR-RbsR and RbsR-GalR. The results are presented in FIGS. 3 and 4. Among mutants with the best performance for each rescued hybrid, there are mutations at three homologous positions, in which some of them are far from the DBM-LBM interface, suggesting that residues at these positions are not directly involved in module-module interactions but they may play key roles in modulating protein confirmation at that interface for facilitating repressor function. These results also revealed the power of coevolutionary coupling analysis in discovering intra-protein interactions.

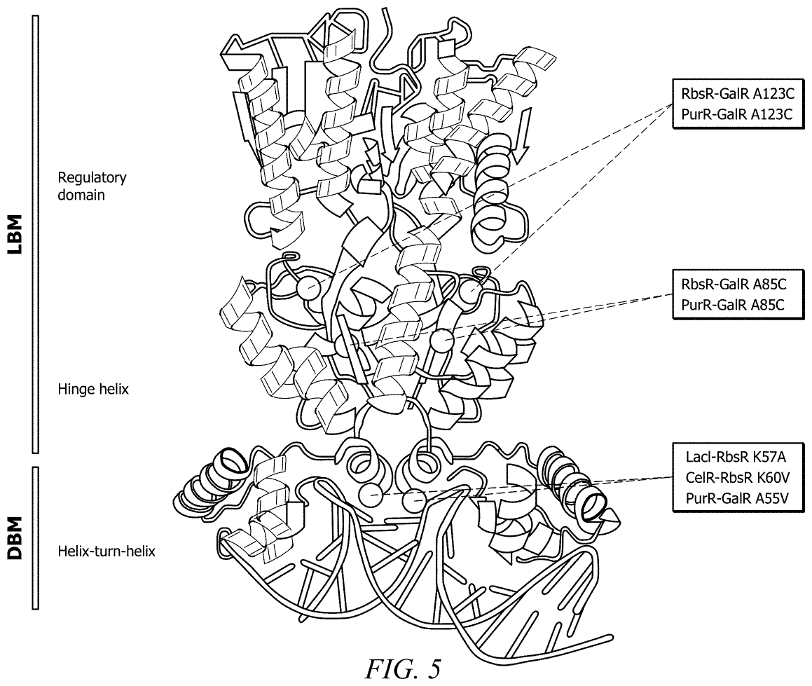

For the original version of these four hybrid repressors, the poor dynamic range of induction can be due to defects in different protein properties—the original LacI-RbsR and CelR-RbsR exhibited high uninduced expression level which indicates weak DNA binding; in contrast, the original PurR-GalR and RbsR-GalR generated low basal expression but repression was not fully released upon induction, suggesting allosteric properties of these repressors were reduced. Intriguingly, the model was successfully used to predict mutations that restore different functions among these repressors. K57 in LacI-RbsR and K60 in CelR-RbsR are homologous position located at the hinge helix motif, FIG. 5 and directly contacting the backbone of DNA but not the nucleobases; it is proposed that the hinge helix is involved in facilitating DNA-protein binding but not recognizing the operator sequence. These results suggest that this position plays a key role in interacting with specific groups of the DNA backbone, such that the DNA and LBM reach a desirable orientation for forming a complex. For the other two rescued repressors, A85/A123 in PurR-GalR and A87C/A126 in RbsR-GalR are distal to DNA and more likely to be involved in a role at inducing allosteric response only. These results strongly imply that disruption of different residue pairs for DBM-LBM interactions can have specific influence on DNA binding and allosteric response.

On the other hand, it was observed that hybrid repressors with similar functional defects can be caused by disruption of different interacting pairs. For PurR-GalR and RbsR-GalR, the two hybrids are structurally similar as they both contain a GalR LBM. The original version of these two repressors also performed similarly, in which both bound tightly to DNA but did not release efficiently from the promoter upon induction. It was first hypothesized that these two hybrid repressors had lost a homologous module-module interacting pair. However, experimental characterization showed that different mutations are required for rescuing the two repressors. PurR-GalR needed three mutations to reach its best performance (245-fold induction), including A55V, A85C, and A123C. Using only the mutants A85C and A123C, there was only 12-fold induction in expression, suggesting that A55V was critical. However, RbsR-GalR gained an improvement of induction fold-change to 69 folds with only A87C and A126C, which are homologous to PurR-GalR's A85C and A123C, respectively. These results suggested that there are no universal sites that are able to rescuing repressors; they form a complex network of interactions that can only be revealed with a global model and metric such as the MSS introduced here.

Folding and structural constraints on highly compatible mutants: While four hybrid repressors were rescued, the performance of another four repressors were not improved based on the MSS. Moreover, some repressors' activities were enhanced with one or two mutations, but not with triple mutations, even though it was inferred that the triple mutant versions would have more favorable compatibility scores. For instance, the original RbsR-GalR generated a 3-fold induction in GFP fluorescence in response to the inducer, galactose; a single mutation A87C or two mutations, A87C and A126C, enhanced the induction to 43-fold and 69-fold, respectively; from the coevolutionary model, the compatibility scores are −69.08 for RbsR-GalR A87C and −71.93 for RbsR-GalR A87C/A126C. A third mutation on RbsR-GalR (G67T or A126C) was expected to further enhance the compatibility between the DBM of RbsR and LBM of GalR; however, the resulting mutants, G69T/A87C/H125M and A87C/H125M/A126C, were inactive, generating induced fold-change of 1.0 and 1.4, respectively. On the other hand, K57A in LacI-RbsR improved the induced fold-change level to 260-fold from an original 6-fold induction, while a double mutation containing K57A and N163Q, and a triple mutation of K57A/F75G/K293F resulted in relatively lower induction levels, 169.6-fold and 146.9-fold, respectively. These results suggested that residues at some positions may have multiple molecular roles; while the model identified that they are involved in DBM-LBM interactions, they may be critical for maintaining structure and function within the LBM, in which mutating these residues leads to a loss of protein function. An original compatibility model only took into consideration of coevolutionary cues between inter-domain residue pairs and did not examine residue pairs within each module. Therefore, mutants designed by using MSS did not evaluate the structure and function of resulting LBMs.

In order to improve the model for the design of hybrid repressors with high induction, it is necessary to further understand molecular interactions within LBMs. For this purpose, an additional metric was introduced into the model, which is based on residue proximity and coevolutionary traits within LBMs. A structure-based score, SF(S), was computed by combining coevolutionary strength between residues within the LBM, with residue-residue distance below 10 Å in a LacI X-ray crystal structure. Similar to the C(S) score, a mutant with increased (more positive) SF(S) is considered as less structurally stable and it may not maintain its protein function.

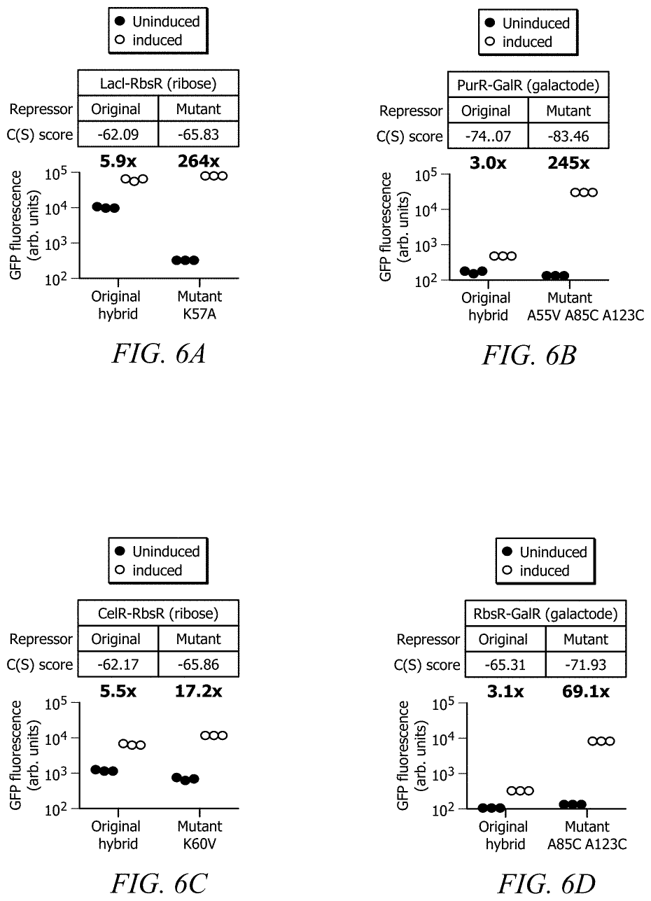

It was then investigated whether an SF(S) can serve as a selection tool to eliminate mutations that lead to a loss of repressor activities. Among all mutants of LacI-RbsR (FIG. 6a), only the K57A mutant has a more favorable SF(S) score compared to its original repressor and indeed, it had the best performance (245-fold induction). Two additional LacI-RbsR mutants are also significantly improved, including K57A/N163Q (170-fold) and K57A/F75G/K293F (147-fold), and their SF(S) scores rank number 2 and number 4, respectively.

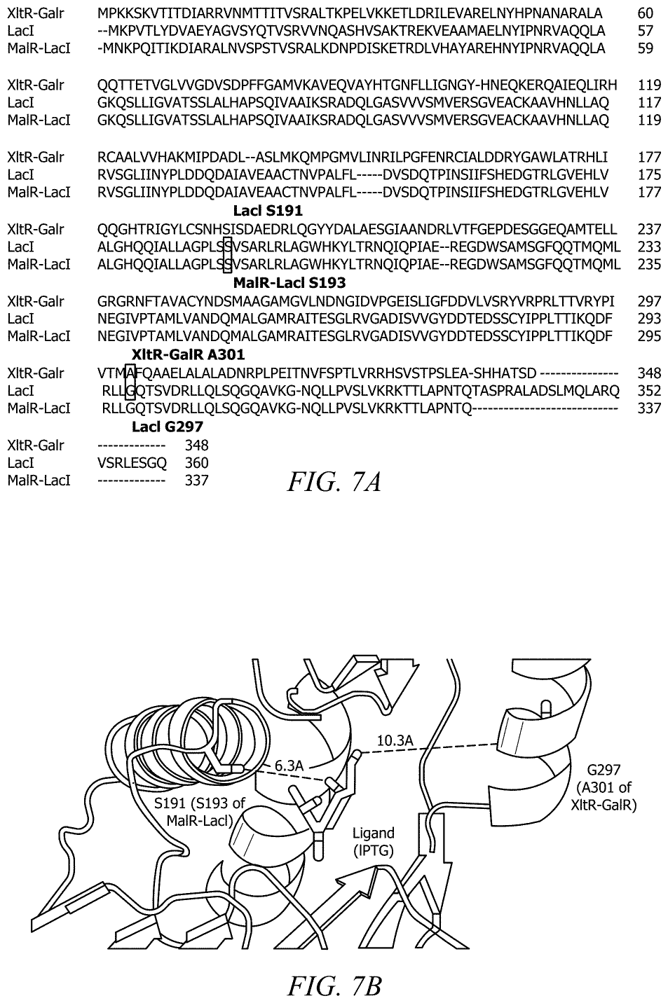

Similarly, for PurR-GalR (FIG. 6b), CelR-RbsR (FIG. 6c), and RbsR-GalR (FIG. 6d), the mutant with the best performance has a SF(S) score better than its original protein. In total, 11 mutants from these four hybrid repressors have a SF(S) score better than the original and 10 of them have fold-induction improved to above 10. In contrast, among those other four hybrid repressors that have not been rescued, including, RbsR-LacI (FIG. 6e), XltR-GalR (FIG. 6f), MalR-LacI (FIG.6g), and XltR-ScrR (FIG. 6h), a majority of their mutants have an SF(S) score worse than their original repressor, indicating that these mutants may not be functional due to possible negative structural effects within the LBM. The few exceptions include MalR-LacI S193I, XltR-GalR A301G, and XltR-GalR E226L/A301G, which have improved SF(S) scores but they remained poorly functional; based upon the crystal structure of LacI, the homologous residues at these positions (S193 of MalR-LacI and A301 of XltR-GalR) are at close proximity to the ligand-binding pocket and mutating them may directly interrupt ligand-binding (FIG. 7), which provided a plausible explanation on the poor functionality of these three mutants. To enhance the robustness of the computational tool, mutations in the ligand-binding pocket could be prohibited. In the current model, mutations in the DNA-binding module were not considered because many residues there are highly involved in interacting with the DNA operator. Similarly, all mutations that are interacting with the ligand directly could be eliminated. Overall, these results strongly support that the SF(S) score reliably indicates whether an LBM mutation is expected to negatively affect repressor activity.

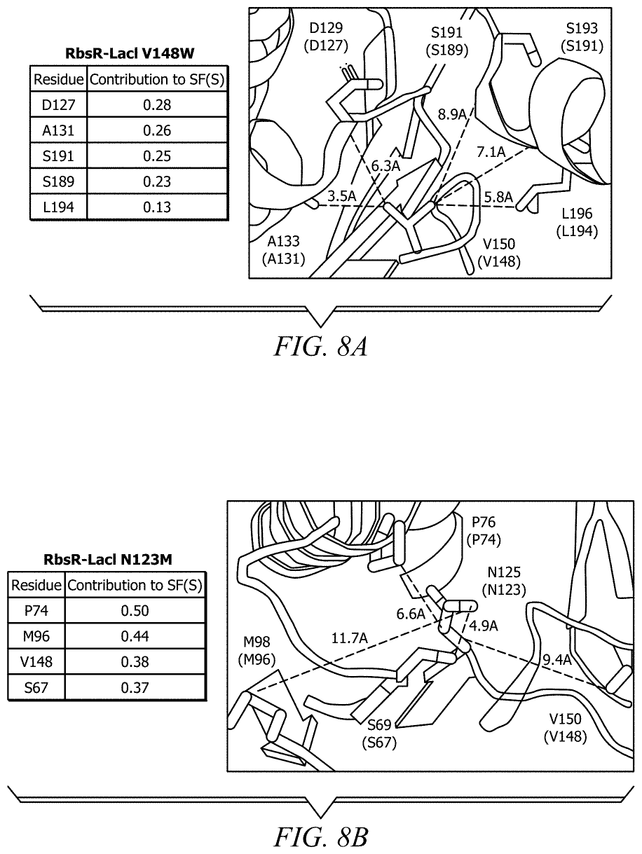

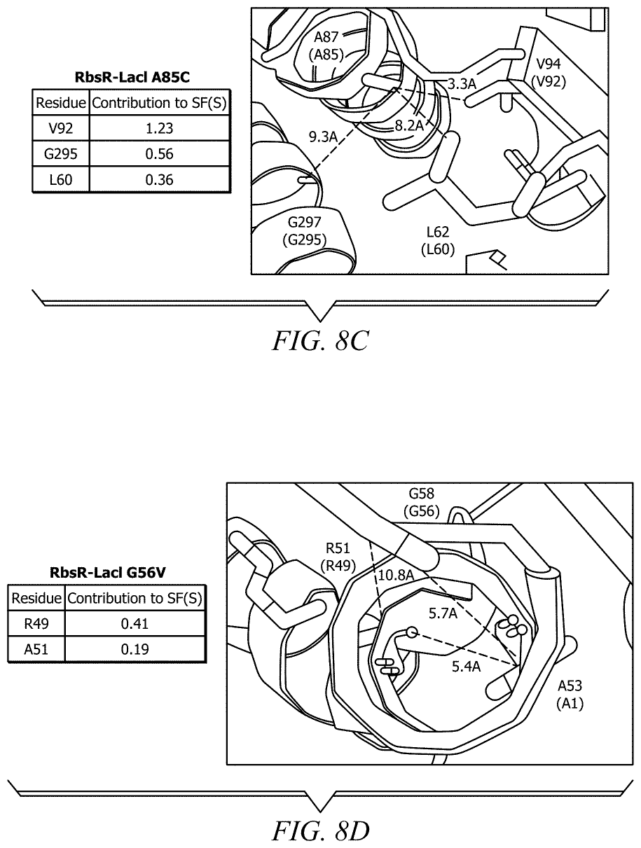

After evaluating the capability of the SF(S) model for predicting mutant performance, this model and a native LacI crystal structure were used to understand how some mutations may disturb intra-module interactions. Taking the case of RbsR-LacI as an example, all four mutations do not improve the repressor function and all of them lead to less favorable SF(S) scores (FIG. 6e). interacting partners of these four mutations were identified from the model, which are major contributors to the change of the SF(S) score (FIG. 8). By studying these identified residues, insights were gained into how these mutations may affect protein function. For mutation V148W in RbsR-LacI (FIG. 8a), valine 148 is surrounded by hydrophobic residues, such as A131 and L194; mutating V148 to a tryptophan may interrupt the hydrophobic interactions between these positions. Additionally, from the crystal structure, several polar residues are at close proximity to V148 but they face the opposition direction, including D127, S191, and S189; the mutation V148W may lead to new hydrogen bonds between the tryptophan and these polar residues, which can trigger significant change in protein confirmation. For mutation N123M (FIG. 8b), N123 is likely to form a strong hydrogen bond with S67, which can be disrupted when mutated to a methionine. For A85C (FIG. 8c), A85 is located at an a-helix and it interacts with residues at a neighboring β-sheet, including V92 and L60. Changing A85 to a cysteine may affect the hydrophobic interface, which is likely to destabilize the β-sheet. Finally, mutation G56V (FIG. 8d) is located at the hinge helix, which plays an essential role in transmitting allosteric signal for controlling DNA binding affinity. G56 interacts with two other residues on the hinge helix, R49 and A51, in which G56V may destabilize the hinge helix and affect its functionality.

In addition to protein design, these examples show how an MSS may also use the SF(S) model to facilitate the study of protein mechanism at a molecular level.

Having described various systems and methods herein, certain aspects can include, but are not limited to:

In a first aspect, a method comprises receiving a protein sequence (S) of a hybrid repressor, wherein the hybrid protein sequence comprises a plurality of DNA-binding modules (DBMs) and a plurality of ligand-binding modules (LBMs); determining an original compatibility score C(S), where the compatibility score C is a function of the protein sequence (S); and predicting, based on the compatibility score C, a performance of the hybrid repressor.

A second aspect can include the method of the first aspect, wherein the compatibility score is determined based on an identification of inter-module residue pairs between the plurality of DBMs and the plurality of LBMs that coevolve.

A third aspect can include the method of the first or second aspect, further comprising evaluating the performance of the hybrid repressor.

A fourth aspect can include the method of the third aspect, wherein the performance of the hybrid repressor is evaluated using one or more transcriptional assays.

A fifth aspect can include the method of any one of the first to fourth aspects, further comprising: identifying one or more replacement LBMs within the protein sequence (S), wherein the one or more replacement LBMs are assigned to replace one or more LBMs in the hybrid repressor; determining a second compatibility score for the protein sequence comprising one or more replacement LBMs; and determining that the second compatibility score relative to the original compatibility score.

A sixth aspect can include the method of the fifth aspect, comprising identifying a second hybrid repressor characterized by a second compatibility score that is from about 5 fold to about 500 fold greater than the original compatibility score.

A seventh aspect can include the method of any one of the first to sixth aspects, further comprising: identifying a plurality of replacement LBMs within the protein sequence (S), wherein the plurality of replacement LBMs are assigned to replace one or more LBMs in the hybrid repressor to identify a plurality of mutation protein sequences; determining a plurality of mutation compatibility scores for the plurality of mutation protein sequences; determining that one or more second compatibility scores of the plurality of compatibility scores are improved relative to the compatibility score by from about 5 fold to about 500 fold; and identifying a second protein sequence for a second hybrid repressor using one or more mutation protein sequences of the plurality of mutation protein sequences having the one or more second compatibility scores, wherein the second hybrid repressor demonstrates a greater functionality than the hybrid repressor.

An eighth aspect can include the method of any one of the first to seventh aspects, wherein the compatibility score C(S) is based on inter-modular coevolutionary coupling strength parameters.

A ninth aspect can include the method of any one of the first to eighth aspects, wherein the compatibility score is further based on residue proximity between the plurality of LBMs.

A tenth aspect can include the method of any one of the first to ninth aspects, further comprising: determining a structure-based score SF(S), where the structure-based score SF is a function of the coevolutionary strength between residues, where predicting the performance of the hybrid repressor is further based on the structure-based score SF.

In an eleventh aspect, a method of constructing a hybrid repressor comprises (a) obtaining a repressor having a protein sequence characterized by a DNA-binding module comprising an amino acid sequence wherein the amino acid sequence has at least 30% homology to the LacI family of proteins and a Ligand-binding module (LBM) comprising an amino acid sequence wherein the amino acid sequence has at least 30% homology to the LacI family of proteins; (b) determining an original compatibility score C(S), where the original compatibility score C is a function of the protein sequence (S) and wherein the original compatibility score is based on inter-modular coevolutionary coupling strength parameters; (c) computationally mutating at least one amino acid residue in the LBM of the repressor to generate a hybrid repressor with a mutated LBM; and (d) determining a compatibility score of the hybrid repressor with a mutated LBM wherein the compatibility score is based on inter-modular coevolutionary coupling strength parameters.

A twelfth aspect can include the method of the eleventh aspect, wherein at least two amino acid residues in the hybrid repressor with a mutated LBM are mutated.

A thirteenth aspect can include the method of the eleventh aspect, wherein at least three amino acid residues in the hybrid repressor with a mutated LBM are mutated.

A fourteenth aspect can include the method of the twelfth aspect, comprising determining a compatibility score of the hybrid repressor with a mutated LBM wherein the compatibility score is based on inter-modular coevolutionary coupling strength parameters.

A fifteenth aspect can include the method of the thirteenth aspect, comprising determining a compatibility score of the hybrid repressor with a mutated LBM wherein the compatibility score is based on inter-modular coevolutionary coupling strength parameters.

A sixteenth aspect can include the method of the twelfth aspect, wherein step (c) is carried out a plurality of times to generate a plurality of hybrid repressors with a mutated LBM.

A seventeenth aspect can include the method of the thirteenth aspect, wherein step (c) is carried out a plurality of times to generate a plurality of hybrid repressors with a mutated LBM.

An eighteenth aspect can include the method of any one of the eleventh to seventeenth aspects, further comprising evaluating the performance of the repressor to obtain a base activity.

A nineteenth aspect can include the method of any one of the eleventh to eighteenth aspects, further comprising evaluating the performance of the hybrid repressor with a mutated LBM to obtain a second activity.

A twentieth aspect can include the method of the nineteenth aspect, further comprising identifying hybrid repressors with a mutated LBM having a second activity that is improved by from about 5 fold to about 500 fold when compared to the base activity.

The subject matter having been shown and described, modifications thereof can be made by one skilled in the art without departing from the spirit and teachings of the subject matter. The aspects described herein are exemplary only and are not intended to be limiting. Many variations and modifications of the subject matter disclosed herein are possible and are within the scope of the disclosed subject matter. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the present invention. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

We claim:

1. A method comprising:
receiving a protein sequence (S) of a hybrid repressor, wherein the hybrid protein sequence comprises a plurality of DNA-binding modules (DBMs) and a plurality of ligand-binding modules (LBMs);
determining an original compatibility score C(S), where the compatibility score C is a function of the protein sequence (S); and
predicting, based on the compatibility score C, a performance of the hybrid repressor.

2. The method of claim 1, wherein the compatibility score is determined based on an identification of inter-module residue pairs between the plurality of DBMs and the plurality of LBMs that coevolve.

3. The method of claim 1, further comprising evaluating the performance of the hybrid repressor.

4. The method of claim 3, wherein the performance of the hybrid repressor is evaluated using one or more transcriptional assays.

5. The method of claim 1, further comprising:
identifying one or more replacement LBMs within the protein sequence (S), wherein the one or more replacement LBMs are assigned to replace one or more LBMs in the hybrid repressor;
determining a second compatibility score for the protein sequence comprising one or more replacement LBMs; and
determining that the second compatibility score relative to the original compatibility score.

6. The method of claim 5, comprising identifying a second hybrid repressor characterized by a second compatibility score that is from about 5 fold to about 500 fold greater than the original compatibility score.

7. The method of claim 1, further comprising:
identifying a plurality of replacement LBMs within the protein sequence (S), wherein the plurality of replacement LBMs are assigned to replace one or more LBMs in the hybrid repressor to identify a plurality of mutation protein sequences;
determining a plurality of mutation compatibility scores for the plurality of mutation protein sequences;
determining that one or more second compatibility scores of the plurality of compatibility scores are improved relative to the compatibility score by from about 5 fold to about 500 fold; and
identifying a second protein sequence for a second hybrid repressor using one or more mutation protein sequences of the plurality of mutation protein sequences having the one or more second compatibility scores, wherein the second hybrid repressor demonstrates a greater functionality than the hybrid repressor.

8. The method of claim 1, wherein the compatibility score C(S) is based on inter-modular coevolutionary coupling strength parameters.

9. The method of claim 1, wherein the compatibility score is further based on residue proximity between the plurality of LBMs.

10. The method of any claim 1, further comprising:
determining a structure-based score SF(S), where the structure-based score SF is a function of the coevolutionary strength between residues, where predicting the performance of the hybrid repressor is further based on the structure-based score SF.

11. A method of constructing a hybrid repressor comprising:
(a) obtaining a repressor having a protein sequence characterized by a DNA-binding module comprising an amino acid sequence wherein the amino acid sequence has at least 30% homology to the LacI family of proteins and a Ligand-binding module (LBM) comprising an amino acid sequence wherein the amino acid sequence has at least 30% homology to the LacI family of proteins;
(b) determining an original compatibility score C(S), where the original compatibility score C is a function of the protein sequence (S) and wherein the original compatibility score is based on inter-modular coevolutionary coupling strength parameters;
(c) computationally mutating at least one amino acid residue in the LBM of the repressor to generate a hybrid repressor with a mutated LBM; and
(d) determining a compatibility score of the hybrid repressor with a mutated LBM wherein the compatibility score is based on inter-modular coevolutionary coupling strength parameters.

12. The method of claim 11, wherein at least two amino acid residues in the hybrid repressor with a mutated LBM are mutated.

13. The method of claim 11, wherein at least three amino acid residues in the hybrid repressor with a mutated LBM are mutated.

14. The method of claim 12, comprising determining a compatibility score of the hybrid repressor with a mutated LBM wherein the compatibility score is based on inter-modular coevolutionary coupling strength parameters.

15. The method of claim 13, comprising determining a compatibility score of the hybrid repressor with a mutated LBM wherein the compatibility score is based on inter-modular coevolutionary coupling strength parameters.

16. The method of claim 12, wherein step (c) is carried out a plurality of times to generate a plurality of hybrid repressors with a mutated LBM.

17. The method of step 13, wherein step (c) is carried out a plurality of times to generate a plurality of hybrid repressors with a mutated LBM.

18. The method of claim 11, further comprising evaluating the performance of the repressor to obtain a base activity.

19. The method of claim 11, further comprising evaluating the performance of the hybrid repressor with a mutated LBM to obtain a second activity.

20. The method of claim 19, further comprising identifying hybrid repressors with a mutated LBM having a second activity that is improved by from about 5 fold to about 500 fold when compared to the base activity.
